(19) United States
(12) Patent Application Publication
(10) Pub. No.: US 2023/0065599 A1
(43) Pub. Date: Mar. 2, 2023

(54) AIR PUMPING DEVICE

(71) Applicants: Futaijing Precision Electronics (Yantai) Co., Ltd., Yantai (CN); HON HAI PRECISION INDUSTRY CO., LTD., New Taipei (TW)

(72) Inventor: HAN-CHUNG YU, New Taipei (TW)

(21) Appl. No.: 17/704,285

(22) Filed: Mar. 25, 2022

(30) Foreign Application Priority Data
Sep. 1, 2021 (CN) 202111022412.1

(51) Int. Cl. A01K 63/04; F04B 35/00; F04B 37/10; A61L 9/015; A61L 9/22; A61L 9/20

(52) U.S. Cl. CPC A01K 63/042; F04B 35/00; F04B 37/10; A61L 9/015; A61L 9/22; A61L 9/20; A61L 2209/14; A61L 2209/134

(57) ABSTRACT

An air pumping device providing automatic sterilization and pumping of indrawn air for oxygenation or other purpose comprises a shell with an air inlet hole and an air outlet hole, an electromagnetic assembly, a transmission device, an air pumping module, and a sterilizer element. The transmission device includes a swinging element forth for acting forces in two directions alternatively on the air pumping module, to draw in and pump out sterilized air. The air pumping device has two air pumping modules is also disclosed, the two air pumping modules being arranged symmetrically on either side of the swing element, therefore the swing element creates an indrawing of air and an exhausting of air for every swing.

### CROSS-REFERENCE TO RELATED APPLICATIONS

This application claims priority to China Application No. 202111022412.1, having a filing date of Sep. 1, 2021, filed in China State Intellectual Property Administration, the entire contents of which is hereby incorporate by reference.

### FIELD

The subject matter relates to air purification technologies, and more particularly, to an air pump with filters.

### BACKGROUND

An aquarium contains aquatic animals and plants, it usually has at least one side made of transparent glass or high-strength plastic. An air pump can draw in external air and output filtered air to the aquarium, and oxygen from the air can be continuously pumped into the water for oxygenation. Therefore, the fresh air can be fed into water in the aquarium by the air pumps, the oxygen from the air is dissolved in the water of the aquarium, resulting in improved oxygen level of the aquarium and sufficient oxygen for fish.

However, the air pumped into the water should be sterilized to avoid viruses or bacteria, and the oxygen output from the air pump ought to be automatically controlled.

### SUMMARY

An objective of the present disclosure is achieved by providing an air pumping device comprising:

a shell, comprising a receiving cavity located therein, at least one air inlet hole placed at a side wall thereon, and an air outlet hole placed at a bottom plate thereon;

an electromagnetic assembly, which is arranged in the receiving cavity and adjacent to the at least one air inlet hole;

a transmission device, comprises a magnetic element, a swing element, a rotation shaft, and a first transmission element, wherein the swing element is rotatably mounted to the rotation shaft at an end thereof away from the electromagnetic assembly, and the magnetic element is fixed at the other end of the swing element. The first transmission element is arranged between the magnetic element and the rotation shaft and located on a side face of the swing element for moving together with the swing element;

a first air pumping module positioned on either side of the swing element, comprising a piston and a cylinder, wherein the piston is mounted on the transmission element via a Cardan joint to move with the transmission element and create reciprocating movement in the cylinder, the cylinder having a one-way inlet valve and a one-way outlet valve;

a sterilizer element, which is arranged with an air inlet connected to the one-way outlet valve and an air outlet connected to the air outlet hole;

as the swing element swings back and forth by turning on the rotation shaft, the piston reciprocates in the cylinder for sucking external air into the cylinder via the one-way inlet valve and pumping air in the cylinder out to the sterilizer element via the one-way outlet valve, and the filtered air will be exported through one-way outlet valve of the sterilizer element.

In operation, the electromagnetic assembly, powered by external power supply, generates pulsed magnetic forces for driving the magnetic element at intervals, which leads the swing element to swing back and forth. As the transmission element moves with the swing element, the piston connected to the transmission element reciprocates in the cylinder. When the piston moves out of the cylinder, external air is sucked into the cylinder via the one-way inlet valve, when the piston moves inside the cylinder, the air sucked in is pumped out from the cylinder to the sterilizer element via the one-way out valve.

The at least one air inlet hole provides an air flow path from external air to the receiving cavity, to the air pumping device and into the air pumping module. The air inlet holes also provide a heat dissipation for the electromagnetic assembly, as the electromagnetic assembly is located adjacently.

According to a further aspect of the application, the air pumping device includes a second air pumping module, the first air pumping module and the second pumping module are arranged in mirror symmetry on both sides of the swing element. A second transmission element is arranged accordingly such that when one of the pistons of the first and second air pumping modules moves inside the cylinder, the other one of the pistons moves to the outside of the cylinder.

Therefore, every movement of the swing acts on both air pumping modules, this double action enhances the efficiency of the air pumping device. Two air pumping modules also provide a better mechanical balance, which stabilizes the movement of the swing element.

The application further discloses an air pumping device comprising:

a shell, comprising a receiving cavity located therein, at least one air inlet hole placed at a side wall thereon, and an air outlet hole placed at a bottom plate thereon;

an electromagnetic assembly, which is arranged in the receiving cavity and adjacent to the at least one air inlet hole;

a transmission device, comprising a magnetic element, a swing element, a rotation shaft, a first transmission element, and a second transmission element. Therein, the swing element is rotatably mounted to the rotation shaft at an end thereof away from the electromagnetic assembly, and the magnetic element is fixed at the other end of the swing element, the first and the second transmission elements are arranged between the magnetic element and the rotation shaft and located on both side faces of the swing element for moving together with the swing element;

a first air pumping module and a second air pumping module, positioned on either side of the swing element, each of the two air pumping modules comprises a bellows made of deformable materials, each bellows is arranged with a one-way inlet valve and a one-way outlet valve;

a sterilizer element, which is arranged with an air inlet connected to the one-way outlet valve and an air outlet connected to the air outlet hole;

the bellows of the two air pumping modules are pressed and deformed alternately by the transmission elements as the swing element swings back and forth by turning on the rotation shaft.

In operation, the electromagnetic assembly, powered by external power supply, generates pulsed magnetic forces for driving the magnetic element at intervals, which leads the swing element to swing back and forth. As the transmission elements move with the swing element, the bellows are alternately pressed into deformation by the transmission elements. When one of the bellows is pressed to pump air out via the one-way outlet valve into the sterilizer element, the other one is restored to suck in external air via the one-way inlet valve.

In some cases, a plurality of air inlet holes are arranged on side walls of the shell, which provides an air flow path from the external air to the receiving cavity, to the air pumping device and into the air pumping module. In addition, the air inlet holes can be positioned on portion of the side walls close to the electromagnetic assembly, providing an enhanced heat dissipation for the electromagnetic assembly.

According to a further aspect of the application, the sterilizer element comprises one or more of a negative-ion generator, an ozonator, and a ultraviolet sterilizer, thereby the air pumped out from the air pumping module is sterilized.

According to a further aspect of the application, each of the air pumping modules further includes a base for supporting a cylinder or a bellows, the base is stably mounted to the shell.

According to a further aspect of the application, the base is made of elastic materials such as rubber, which acts as a buffer for the vibration generated during operation of the cylinder/bellows.

According to a further aspect of the application, the electromagnetic assembly comprises a metal core and a coil, the metal core is “U” shaped and one end of the metal core is arranged through the coil. When the coil is energized, the efficiency of converting electric energy into magnetic energy is enhanced, and the utilization efficiency of electric energy is improved.

According to a further aspect of the application, the air pumping device includes a power cable connected from an external power supply to the electromagnetic assembly for powering the electromagnetic assembly, and a power cable connected from external power supply to the sterilizer element for powering the sterilizer element.

### BRIEF DESCRIPTION OF THE DRAWINGS

Implementations of the present technology will now be described, by way of example only, with reference to the attached figures.

FIG. 1 is a perspective view of a first embodiment of an air pump according to the present disclosure mounted on a aquarium.

FIG. 2 is a perspective view of a first embodiment of an air pump according to the present disclosure.

FIG. 3 is a cross section view of the air pump in the first embodiment according to the present disclosure.

FIG. 4 is a perspective view of a second embodiment of an air pump according to the present disclosure.

In the drawings and in the detailed part of the description, the following references are used:
Air pumping device 100
Shell 10
Receiving cavity 11
Air inlet hole 12
Side wall 13
Air outlet hole 14
Bottom plate 15
Electromagnetic assembly 20
Metal core 21
Coil 22
Transmission device 30
Magnetic element 31
Swing element 32
Rotation shaft 33
First transmission element 34
Second transmission element 35
First air pumping module 40
Piston 41
Cylinder 42
One-way inlet valve 422
Outlet conduit 43
Base 44
Cardan joint 45
Bellows 46
Sterilizer element 50
Air inlet 51
Air outlet 52
Air conduit 60
Second air pumping module 70

### DETAILED DESCRIPTION

It will be appreciated that for simplicity and clarity of illustration, where appropriate, reference numerals have been repeated among the different figures to indicate corresponding or analogous components. The description is not to be considered as limiting the scope of the embodiments described herein. The drawings are not necessarily to scale and the proportions of certain parts may be exaggerated to better illustrate details and features of the present disclosure.

It should be understood that, the terms “first” and “second” are used to distinguish elements and are not used to denote a particular order or imply a number of technical features, therefore, unless being specifically defined, features described with “first” and “second” may expressly or implicitly include one or more of the stated features. In the description of the present application, “plurality” means two or more, unless otherwise expressly and specifically defined.

In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described.

The term “comprising,” when utilized, means “including, but not necessarily limited to”; it specifically indicates open-ended inclusion or membership in the so-described combination, group, series, and the like.

An aspect of the present disclosure provides an air pumping device comprising:

a shell, comprising a receiving cavity located therein, at least one air inlet hole placed at a side wall thereon, and an air outlet hole placed at a bottom plate thereon;

an electromagnetic assembly, which is arranged in the receiving cavity and adjacent to the at least one air inlet hole;

a transmission device, comprises a magnetic element, a swing element, a rotation shaft, and a first transmission element, wherein the swing element is rotatably mounted to the rotation shaft at an end thereof away from the electromagnetic assembly, and the magnetic element is fixed at the other end of the swing element. The first transmission element is arranged between the magnetic element and the rotation shaft and located on a side face of the swing element for moving together with the swing element;

a first air pumping module positioned on either side of the swing element, comprising a piston and a cylinder, wherein the piston is mounted on the transmission element via a Cardan joint to move with the transmission element and create reciprocating movement in the cylinder, the cylinder having a one-way inlet valve and a one-way outlet valve;

a sterilizer element, which is arranged with an air inlet connected to the one-way outlet valve and an air outlet connected to the air outlet hole;

as the swing element swings back and forth by turning on the rotation shaft, the piston reciprocates in the cylinder for sucking external air into the cylinder via the one-way inlet valve and pumping air in the cylinder out to the sterilizer element via the one-way outlet valve, and the filtered air will be exported through one-way outlet valve of the sterilizer element.

In operation, the electromagnetic assembly, powered by external power supply, generates pulsed magnetic forces for driving the magnetic element at intervals, which leads the swing element to swing back and forth. As the transmission element moves with the swing element, the piston connected to the transmission element reciprocates in the cylinder. When the piston moves out of the cylinder, external air is sucked into the cylinder via the one-way inlet valve, when the piston moves inside the cylinder, the air sucked in is pumped out from the cylinder to the sterilizer element via the one-way out valve.

The at least one air inlet hole provides an air flow path from external air to the receiving cavity, to the air pumping device and into the air pumping module. The air inlet holes also provide a heat dissipation for the electromagnetic assembly, as the electromagnetic assembly is located adjacently.

According to a further aspect of the application, the air pumping device includes a second air pumping module, the first air pumping module and the second pumping module are arranged in mirror symmetry on both sides of the swing element. A second transmission element is arranged accordingly such that when one of the pistons of the first and second air pumping modules moves inside the cylinder, the other one of the pistons moves to the outside of the cylinder.

Therefore, every movement of the swing acts on both air pumping modules, this double action enhances the efficiency of the air pumping device. Two air pumping modules also provide a better mechanical balance, which stabilizes the movement of the swing element.

The application further discloses an air pumping device comprising:

a shell, comprising a receiving cavity located therein, at least one air inlet hole placed at a side wall thereon, and an air outlet hole placed at a bottom plate thereon;

an electromagnetic assembly, which is arranged in the receiving cavity and adjacent to the at least one air inlet hole;

a transmission device, comprising a magnetic element, a swing element, a rotation shaft, a first transmission element, and a second transmission element. Therein, the swing element is rotatably mounted to the rotation shaft at an end thereof away from the electromagnetic assembly, and the magnetic element is fixed at the other end of the swing element, the first and the second transmission elements are arranged between the magnetic element and the rotation shaft and located on both side faces of the swing element for moving together with the swing element;

a first air pumping module and a second air pumping module, positioned on either side of the swing element, each of the two air pumping modules comprises a bellows made of deformable materials, each bellows is arranged with a one-way inlet valve and a one-way outlet valve;

a sterilizer element, which is arranged with an air inlet connected to the one-way outlet valve and an air outlet connected to the air outlet hole;

the bellows of the two air pumping modules are pressed and deformed alternately by the transmission elements as the swing element swings back and forth by turning on the rotation shaft.

In operation, the electromagnetic assembly, powered by external power supply, generates pulsed magnetic forces for driving the magnetic element at intervals, which leads the swing element to swing back and forth. As the transmission elements move with the swing element, the bellows are alternately pressed into deformation by the transmission elements. When one of the bellows is pressed to pump air out via the one-way outlet valve into the sterilizer element, the other one is restored to suck in external air via the one-way inlet valve.

In some cases, a plurality of air inlet holes are arranged on side walls of the shell, which provides an air flow path from the external air to the receiving cavity, to the air pumping device and into the air pumping module. In addition, the air inlet holes can be positioned on portion of the side walls close to the electromagnetic assembly, providing an enhanced heat dissipation for the electromagnetic assembly.

According to a further aspect of the application, the sterilizer element comprises one or more of a negative-ion generator, an ozonator, and a ultraviolet sterilizer, thereby the air pumped out from the air pumping module is sterilized.

According to a further aspect of the application, each of the air pumping modules further includes a base for supporting a cylinder or a bellows, the base is stably mounted to the shell.

According to a further aspect of the application, the base is made of elastic materials such as rubber, which acts as a buffer for the vibration generated during operation of the cylinder/bellows.

According to a further aspect of the application, the electromagnetic assembly comprises a metal core and a coil, the metal core is “U” shaped and one end of the metal core is arranged through the coil. When the coil is energized, the efficiency of converting electric energy into magnetic energy is enhanced, and the utilization efficiency of electric energy is improved.

According to a further aspect of the application, the air pumping device includes a power cable connected from an external power supply to the electromagnetic assembly for powering the electromagnetic assembly, and a power cable connected from external power supply to the sterilizer element for powering the sterilizer element.

A detailed description of the hereinafter described embodiments of the disclosure is presented herein by way of exemplification and not limitation with reference to the figures.

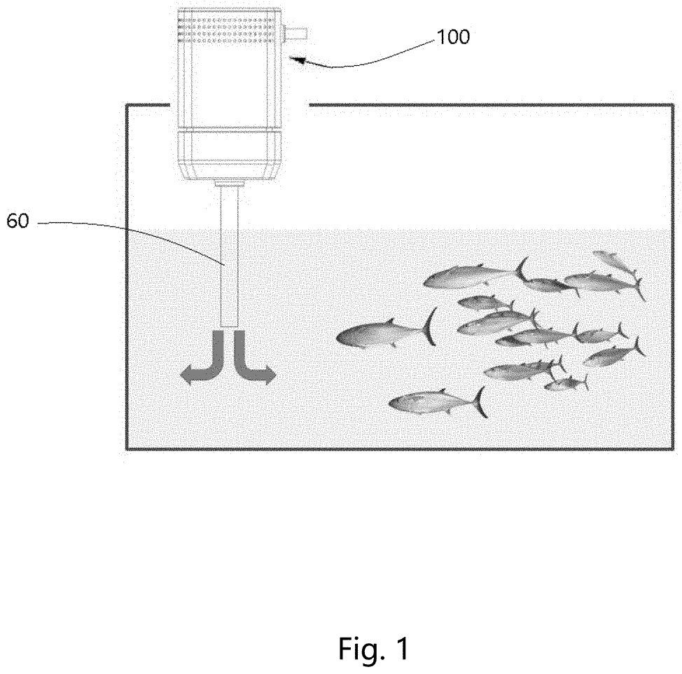

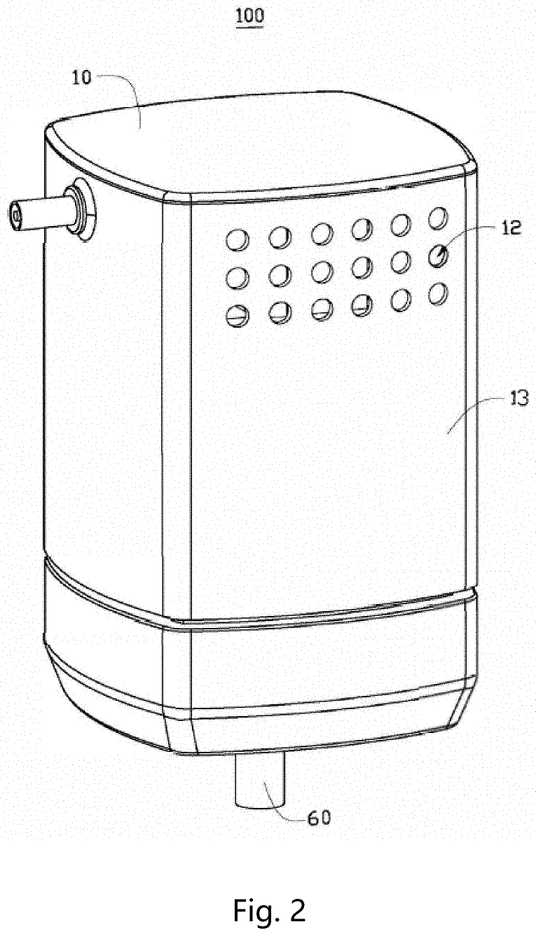

Referring to FIGS. 1-3, an air pumping device 100 is provided by a first embodiment of the disclosure, the air pumping device 100 includes a shell 10 comprising a receiving cavity 11 located therein. An electromagnetic assembly 20, a transmission device 30, a first air pumping module 40, a sterilizer element 50, and a second air pumping module 70 are arranged in the receiving cavity 11. The transmission assembly 30, driven by the electromagnetic assembly 20, moves air from the first air pumping module 40 and second air pumping module 70 to the sterilizer element 50, then the air is sterilized and pumped out from the sterilizer element 50.

Specifically, the shell is arranged with at least one air inlet hole 12 placed at a side wall 13 thereon and an air outlet hole 14 placed at a bottom plate 15 thereon. The transmission assembly 30 comprises a magnetic element 31, a swing element 32, a rotation shaft 33, a first transmission element 34, and a second transmission element 35, wherein the swing element 32 is rotatably mounted to the rotation shaft 33 at an end thereof away from the electromagnetic assembly 20 and the magnetic element 31 is fixed at the other end of the magnetic element 31. The first transmission element 34 and the second transmission element 35 are arranged between the magnetic element 31 and the rotation shaft 33 and located on each side of the swing element 32 for moving together with the swing element 32.

The first air pumping module 40 and the second air pumping module 70 are located symmetrically on either side of the swing element 32. Each of the first air pumping module 40 and the second air pumping module 70 includes a piston 41 and a cylinder 42, wherein the piston 41 is mounted on the transmission element 34 via a Cardan joint 45 to move with the transmission element 34 for reciprocating in the cylinder 42. Each cylinder 42 is arranged with a one-way inlet valve 421 and a one-way outlet valve 422 for air suction and air exhausting. The sterilizer element 50 is arranged with an air inlet 51 connected to the one-way outlet valve 422 and an air outlet 52 connected to the air outlet hole 13.

In operation, the electromagnetic assembly 20, powered by external power supply, generates pulsed magnetic forces for driving the magnetic element 31 at intervals, which leads the swing element 32 to swing back and forth. As the first transmission element 34 and the second transmission element 35 move with the swing element 32, the pistons 41 connected to the transmission element reciprocate in their cylinders 42. When one of the pistons 41 is moving towards the respective cylinder 42, the other one is moving away from the respective cylinder 42.

In each air pumping module, external air enters the cylinder 42 via the one-way inlet valve 421 when the piston 41 moves away from the cylinder 42 and being pumped out from the cylinder 42 into the outlet conduit 43 via the one-way outlet valve 422 when the piston 41 moves towards the cylinder 42. After being pumped out from the cylinder 42, the air enters the sterilizer element 50 for sterilization from the outlet conduit 43 via an air inlet 51, then the filtered air is expelled via an air outlet 52, and thereby filtered air is pumped into the water in the aquarium, oxygen level of the aquarium can be improved and sufficient oxygen is provided for fish growing.

In this embodiment, the electromagnetic assembly 20 includes a metal core 21 and a coil 22, the metal core 21 is “U” shaped and one end thereof is arranged through the coil 22. The magnetic element 31 is a permanent magnet, the swing element 32 is a rectangular-section pole, and the rotation shaft 33 is a circular-section pole for rotating the swing element 32. By forming the electromagnetic assembly 20 with the metal core 21 and the coil 22, when the coil 22 is energized, the efficiency of converting electric energy into magnetic energy is enhanced, and the utilization efficiency of electric energy is improved.

According to the application, the sterilizer element 50 comprises one or more of a negative-ion generator, an ozonator, and an ultraviolet sterilizer. In this embodiment, the sterilizer element 50 comprises a negative-ion generator that is electrically connected to an external power supply, the negative-ion generator generates negative-ions and minute quantities of ozone, which kills viruses and bacteria, thereby the air pumped from the air pumping module 40 is sterilized.

According to the application, the air pumping device 100 includes a power cable, which connects from external power supply to the electromagnetic assembly 20 for powering the electromagnetic assembly 20, and a power cable connected from external power supply to the sterilizer element 50 for powering the sterilizer element 50.

In a preferred embodiment, the shell 10 is configured with a plurality of air inlet holes 12 on opposite side walls of the shell, which provide air flow path from the external air to the air pumping device and to the air pumping module 40.

In a preferred embodiment, the electromagnetic assembly 20 is arranged adjacent to the air inlet holes 12 on the side walls, thereby the electromagnetic assembly 20 is provided with a better heat dissipation.

In a preferred embodiment, the air pumping device 100 includes an air conduit 60, the air conduit 60 is connected to the air outlet 52 at one end and extends through the air outlet hole 13 and into the water in the aquarium, therefore, filtered air is pumped into the water.

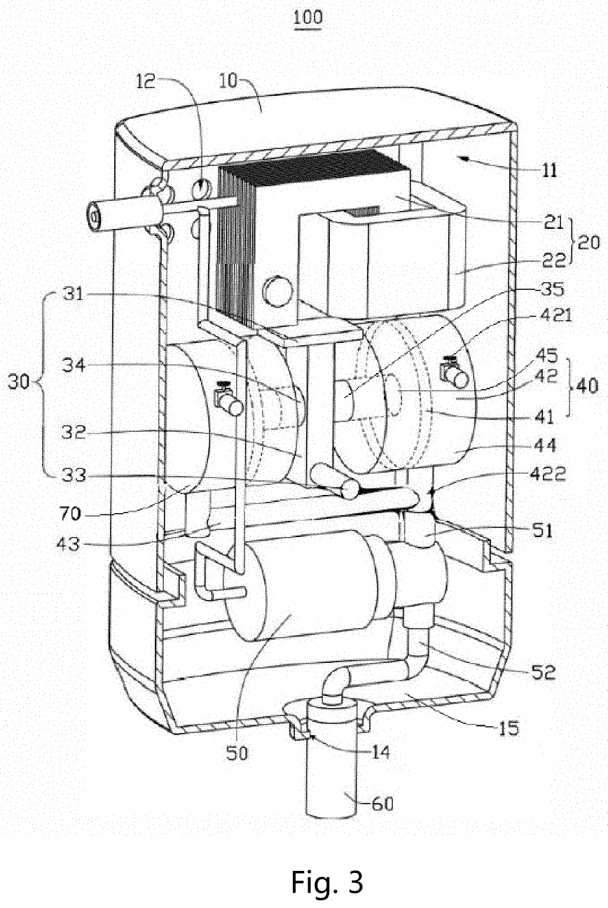

As shown in FIG. 3, in a preferred embodiment, the air pumping module 40 includes a strong and stable base 44 for supporting the cylinder 42, the base 44 is mounted to the shell 10. Therefore, the cylinder 42 can be more steady in operation.

In a preferred embodiment, the base 44 is made of elastic materials such as rubber, which acts as a buffer for vibration generated during operation of the cylinder 42.

As seen in FIGS. 1-3, in operation, the air pumping device 100 is mounted on an aquarium with the air conduit 60 insert into the water, the electromagnetic assembly 20 is powered by external power supply to generate pulsed magnetic force for forcing away the magnetic element 31 at intervals, then the swing element to turns on the rotation shaft 33, which drives the transmission elements 34 and the pistons 41 thereon to reciprocate in their respective cylinders 42. Therefore, the external air is cyclically sucked into the two cylinders 42 of the first air pumping module 40 and the second air pumping module 70 and pumped into the sterilizer element 50 for sterilization, and after sterilization, the filtered air enters water in the aquarium via the air outlet 52, thereby filtered air is pumped into the water in the aquarium, oxygen level of the aquarium can be improved and sufficient oxygen is provided for fish growing.

In a preferred embodiment, the air pumping device 100 is arranged with only one air pumping module, that is, the second air pumping module 70 and the second transmission element 35 are removed, which provides more space for the swing element 32 and allows a greater amplitude of swing. Therefore, the air pumping device provides a greater capacity of one air pumping module for each operation cycle, and the size of the air pumping device is reduced.

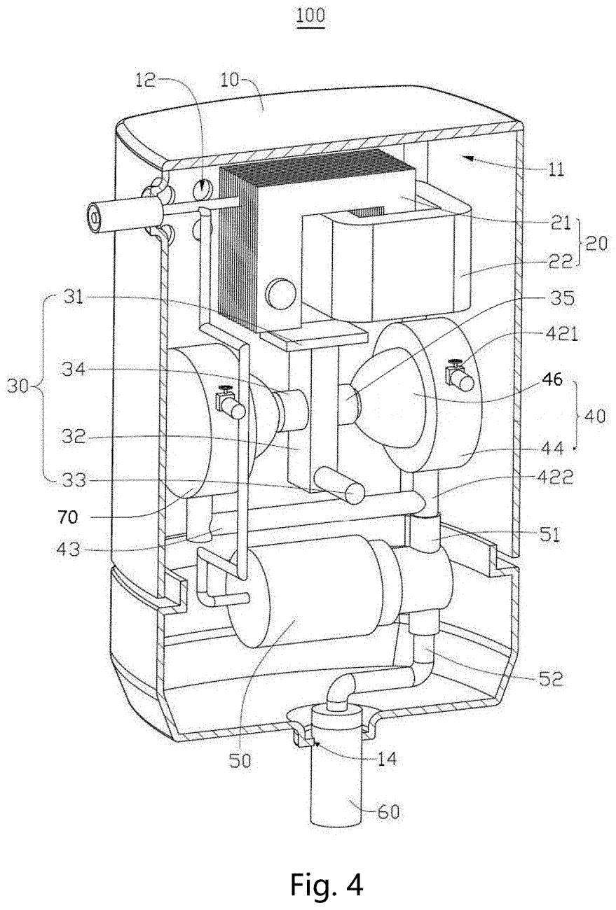

As seen in FIG. 4, in a further embodiment, each of the first air pumping module 40 and the second air pumping module 70 is arranged with a hemispherical bellows 46 made of deformable materials such as rubber, the cylinders and pistons are removed. The external air is cyclically sucked into the two bellows 46 and pumped into the sterilizer element 50 as the swing element 32 swings forth and back to deform the two cylinders alternately.

While the invention has been described in detail in connection with only a limited number of embodiments, it should be readily understood for the skilled in the art that the invention is not limited to such disclosed embodiments. Rather, the invention can be modified to incorporate any number of variations, alterations, substitutions, or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. An air pumping device comprising:
a shell comprising a receiving cavity located therein, at least one air inlet hole placed at a side wall thereon, and an air outlet hole placed at a bottom plate thereon;
an electromagnetic assembly arranged in the receiving cavity and adjacent to the at least one air inlet hole;
a transmission device comprising a magnetic element, a swing element, a rotation shaft and a first transmission element, wherein the swing element is rotatably mounted to the rotation shaft at an end thereof away from the electromagnetic assembly, and the magnetic element is fixed at the other end of the swing element, the first transmission element is arranged between the magnetic element and the rotation shaft and located on a side face of the swing element for moving together with the swing element;
a first air pumping module positioned on either side of the swing element, the first air pumping module comprising a piston and a cylinder, wherein the piston is mounted on the transmission element via a cardan join to move with the transmission element for reciprocating in the cylinder; the cylinder is arranged with a one-way inlet valve and a one-way outlet valve; and
a sterilizer element arranged with an air inlet connected to the one-way outlet valve and an air outlet connected to the air outlet hole;
wherein as the swing element swings back and forth by turning on the rotation shaft, the piston reciprocates in the cylinder for sucking external air into the cylinder via the one-way inlet valve and pump air in the cylinder out to the sterilizer element via the one-way out valve, and the filtered air will be exported through one-way outlet valve of the sterilizer element.

2. The air pumping device of claim 1, further comprising a second air pumping module, wherein the first air pumping module and the second pumping module are arranged mirror symmetry on both sides of the swing element, a second transmission element is arranged accordingly, when one of the pistons of the first and second air pumping modules moves towards the respective cylinder, the other one of the pistons moves away from the respective cylinder.

3. An air pumping device comprising:
a shell comprising a receiving cavity located therein, at least one air inlet hole placed at a side wall thereon, and an air outlet hole placed at a bottom plate thereon;
an electromagnetic assembly arranged in the receiving cavity and adjacent to the at least one air inlet hole;
a transmission device comprising a magnetic element, a swing element, a rotation shaft a first transmission element, and a second transmission element, wherein the swing element is rotatably mounted to the rotation shaft at an end thereof away from the electromagnetic assembly, and the magnetic element is fixed at the other end of the swing element, the first and the second transmission elements are arranged between the magnetic element and the rotation shaft and located on both side faces of the swing element for moving together with the swing element;
a first air pumping module and a second air pumping module, positioned on both sides of the swing element, respectively, each of the two air pumping modules comprising a bellows made of deformable materials, each bellows is arranged with a one-way inlet valve and a one-way outlet valve; and
a sterilizer element arranged with an air inlet connected to the one-way outlet valve and an air outlet connected to the air outlet hole;
wherein the cylinders of the two air pumping modules are pressed by the transmission elements for deformation alternatively as the swing element swings back and forth by turning on the rotation shaft.

4. The air pumping device of claim 1, wherein the sterilizer element comprises one or more of a negative-ion generator, an ozonator and a ultraviolet sterilizer.

5. The air pumping device of claim 2, wherein the sterilizer element comprises one or more of a negative-ion generator, an ozonator and a ultraviolet sterilizer.

6. The air pumping device of claim 3, wherein the sterilizer element comprises one or more of a negative-ion generator, an ozonator and a ultraviolet sterilizer.

7. The air pumping device of claim 1, wherein each of the first and second air pumping modules further comprises a base configured for supporting the respective cylinder, the base is mounted to the shell.

8. The air pumping device of claim 2, wherein each of the first and second air pumping modules further comprises a base configured for supporting the respective cylinder, the base is mounted to the shell.

9. The air pumping device of claim 3, wherein each of the first and second air pumping modules further comprises a base configured for supporting the respective bellows, the base is mounted to the shell.

10. The air pumping device of claim 7, wherein the base is made of elastic materials.

11. The air pumping device of claim 8, wherein the base is made of elastic materials.

12. The air pumping device of claim 9, wherein the base is made of elastic materials.

13. The air pumping device of claim 1, wherein the electromagnetic assembly comprises a metal core and a coil, the metal core is “U” shaped and one end of the metal core is arranged through the coil.

14. The air pumping device of claim 2, wherein the electromagnetic assembly comprises a metal core and a coil, the metal core is “U” shaped and one end of the metal core is arranged through the coil.

15. The air pumping device of claim 3, wherein the electromagnetic assembly comprises a metal core and a coil, the metal core is “U” shaped and one end of the metal core is arranged through the coil.
